(19) United States
(12) Patent Application Publication
(10) Pub. No.: US 2006/0019935 A1
(43) Pub. Date: Jan. 26, 2006

(54) Composition and method for treating acne including anti-inflammatory Hepes Oleate

(76) Inventor: Dana M. Watson, Raleigh, NC (US)

Correspondence Address: H. JAY SPIEGEL, P.O. BOX 11, Mount Vernon, VA, 22121, US

(21) Appl. No.: 10/894,047

(22) Filed: Jul. 20, 2004

(51) Int. Cl. A61K 31/573; A61K 31/22

(52) U.S. Cl. 514/171; 514/546

(57) ABSTRACT

The present invention is directed toward a topical composition for the treatment of acne comprising water, an anti-acne agent such as benzoyl peroxide, thickening agent, emulsifiers, stabilizers, and a natural esterified anti-inflammatory (Hepes Oleate). This combined therapy is more effective than either active ingredient alone and is particularly effective for those who do not respond well to benzoyl peroxide alone.

### BACKGROUND OF INVENTION

The present invention relates to topical compositions for the treatment of acne and inflammatory acne including, as an active ingredient, an anti-inflammatory agent known as Hepes Oleate, and the method of treatment employing such compositions.

Acne and seborrhea are conditions of the skin characterized by an excessive flow of sebum from the sebaceous glands. Sebum reaches the skin surface through the duct of the hair follicle. The presence of excessive amounts of sebum in the duct, along with excessive shedding of the skin cells inside the duct, and in the skin acts to block the continuous flow of sebum from the follicular duct. This produces a thickening of the sebum which becomes a comedone. Comedone formation is followed by hyperkeratinization of the follicular opening and thus closing the duct completely. This results in a papule, a pustule, or a cyst, often contaminated with bacteria which cause secondary infections and associated with inflammation.

There are many topical agents used in the treatment of acne and seborrhea to prevent the blocking of the follicular duct, to reopen the duct once it has been clogged and to act against the infecting bacteria.

Several anti-acne agents are well known in the art. These include, for example, benzoyl peroxide, alpha hydroxy acids and detergents. Benzoyl peroxide is a colorless, odorless, tasteless crystalline solid that is stable at ordinary room temperatures. It is also a strong oxidizing agent which may be used as an antibacterial agent in treating acne. Due to the fact that benzoyl peroxide has such strong oxidizing properties, inclusion of it in conventional creams often results in unstable compositions and a rapid loss of potency.

A stable benzoyl peroxide composition that has been very effective in the treatment of acne and has a projected shelf life of eight years is described in U.S. Pat. No. 3,535,422 to Cox et al. This patent describes a uniform dispersion of fine benzoyl peroxide particles in an emulsion of water and certain organic emollients. When this composition is applied to the skin, the water content of the emulsion evaporates which leaves most of the organic emollients and the benzoyl peroxide particles on the surface of the skin and in contact with the acne sites.

Benzoyl peroxide has been reported to be irritating to the skin when applied at concentrations appropriate for the treatment of acne. Consequently, anti-acne compositions containing benzoyl peroxide often contain one or more moisturizers in order to minimize skin irritation associated with an anti-acne agent.

U.S. Pat. No. 6,737,070 discloses a relation to the use of transitional metals Cu (1) and ferrous ions to increase the efficacy of peroxides such as benzoyl peroxide.

U.S. Pat. No. 5,733,886 discloses a suitable treatment for acne combining clindamyacin and benzoyl peroxide in separate containers for the promotion of stability.

U.S. Pat. No. 6,433,024 discloses a topical composition for treating acne comprising of water, organic peroxide, and alpha hydroxy acid, a moisturizer, an isoorbide and a detergent.

None of the compositions of the aforementioned references provide a benzoyl peroxide composition which combines the desired anti-acne properties with the enhanced stabilization of adding a reducing agent, such as hydrocortisone, for additional stability of the organic peroxide and the inclusion of an anti-inflammatory agent to reduce the swelling, redness, and pain associated with inflamed acne.

U.S. Pat. No. 6,114,337 to Pugliese et al. discloses the synthesis of Hepes Oleate and discloses this substance as having anti-inflammatory properties. Pugliese et al. disclose the use of such substances in conjunction with topically applied products so as to reduce symptoms of skin inflammation wherein the particular etiology of the inflammation does not call for or require the use of antibiotics or germicidal compositions. Pugliese et al. also suggest using Hepes-based compounds in association with formulations for epidermal penetration on bruises, muscle strains, and sprains. They also suggest the use of Hepes esters as a cosmetic formulation ingredient, as a co-emulsifier usable with topical analgesics as well as in cosmetic preparations in conjunction with active ingredients intended to retard skin aging. Pugliese et al. fail to teach or suggest employing an anti-inflammatory agent such as Hepes Oleate with an acne medication nor do they teach or suggest the synergy discovered by Applicant that enhances the acne fighting capability and anti-inflammatory powers when the Hepes Oleate is combined with acne medication in accordance with the teachings of the present invention, wherein the pH of the Hepes Oleate closely matches that of the skin to preclude stinging of the skin during application.

U.S. Pat. Nos. 4,544,656 and 4,753,942 teach use of Hepes, Ethane Sulphonic Acid topically to treat arthritis and rheumatis as well as psoriasis. These patents do not teach or suggest use of a Hepes ester such as Hepes Oleate.

An object of the present invention is to provide a stable topical composition for the treatment of acne that is less irritating, and will reduce the inflammation associated with papulo-pustular inflammatory acne. It would also be advantageous if the anti-inflammatory agent were available over the counter so that the acne treatment could be purchased without the need for a prescription.

### SUMMARY OF INVENTION

The present invention relates to topical compositions for treatment of acne and inflammatory acne and the method of treatment employing such compositions. The present invention includes the following interrelated objects, aspects and features:

(1) According to the present invention, mild to severe (to include papulo-pustular inflammatory acne and cystic acne) acne can be controlled and cleared more effectively than with the use of topical anti-acne compositions in the prior art by administering to the affected areas of the skin a composition comprising water, benzoyl peroxide, thickening agent, emulsifiers, stabilizers, and a new chemical entity that blocks the inflammatory cascade at the level of phospholipase A₂ (Hepes Oleate).

(2) The benzoyl peroxide and the Hepes compound have a synergistic effect when in a single formulation which leads to a more rapid clearing and is notably effective in the treatment of acne and inflammatory acne. Typically acne can be controlled and cleared by once or twice daily applications of a composition containing water, benzoyl peroxide, thickening agent, emulsifiers, stabilizers, and an anti-inflammatory (Hepes Oleate).

(3) In the preferred embodiment of the present invention, the inventive composition includes from about 1% to 20%, by weight, acne medication such as benzoyl peroxide, and from about 0.5% to 20%, by weight, Hepes Oleate.

(4) The inventive composition preferably includes from about 25% to 70%, by weight, deionized water, as well as a moisturizer, emulsifier, and, if necessary, thickeners.

(5) A pH balancer may also be employed so that the composition does not sting the skin when applied and anti-microbials and preservatives may also be included.

(6) Once the inventive composition has succeeded in clearing the skin of acne while reducing inflammation, clearance can be maintained by less frequent and/or less potent applications of this composition.

As such, it is a first object of the present invention to provide a topical composition for the treatment of acne while reducing inflammation of the skin.

It is a further object of the present invention to provide such a composition in which the active ingredients include benzoyl peroxide and Hepes Oleate.

It is a further object of the present invention to provide such a composition including a moisturizer, an emulsifier, and thickeners.

It is a still further object of the present invention to provide such a composition including a pH balancer, anti-microbials and preservatives.

It is a yet further object of the present invention to provide a method of treating the skin employing the inventive composition.

These and other objects, aspects and features of the present invention will be better understood from the following detailed description of the preferred embodiments.

### SPECIFIC DESCRIPTION OF THE PREFERRED EMBODIMENTS

Benzoyl peroxide is a strong oxidizing agent which may be used in topical compositions for treating acne. However, when applied in concentrations that are effective for treating acne, benzoyl peroxide also may be irritating. Consequently, many compositions that contain benzoyl peroxide also include one or more moisturizers. Unfortunately, moisturizers often interfere with benzoyl peroxide's ability to be in contact with the skin and thereby reduce its effectiveness in treating acne. This will often occur with oil-based moisturizers, such as isopropyl myristate, mineral oil, or petrolatum. Additionally, benzoyl peroxide does not provide any anti-inflammatory properties.

The present invention is based in part on the use of a strong reducing agent as a stabilizer for the benzoyl peroxide combined with an oil-based moisturizer or water based moisturizer. A strong reducing and stabilizing agent, such as hydrocortisone, in a small proportion that does not result in the negative effects of a steroid, will reduce the oxidation of a strong oxidizing agent such as, benzoyl peroxide. This composition will provide a moisturizing base for the benzoyl peroxide without a significant loss of efficacy and without interfering with benzoyl peroxide's ability to be in contact with the skin.

The present invention is based also in part on the use of a non-steroidal, anti-inflammatory agent. This composition comprising water, benzoyl peroxide, thickening agent, emulsifiers, stabilizers, and an anti-inflammatory (Hepes Oleate) has a significant effect, resulting in the reduction of swelling, relieving sensitivity and redness associated with inflammatory acne conditions. Any inflammation that occurs in the body is the end result of a series of events known as the arachidonic acid cascade. The process starts with a traumatic or chemical event that leads to injury of the cell membrane. Cell membranes consist of phosolipids, which are complex lipid materials that contain fatty acids, one of which is arachidonic acid. In the initiation of the inflammatory process the first step is the conversion of arachidonic acid into the specific mediators of inflammation, which follows two pathways. One of the pathways is called the cyclooxygenase pathway and the other is called the lipoxygenase pathway. All anti-inflammatory agents block the formation of the end products of these two pathways. Aspirin for example blocks the cyclooxygenase pathway, while cortisone block both pathways by interfering with the formation of arachidonic acid. HO-1 (Hepes Oleate) works in the same fashion, blocking both pathways and interfering with the formation of arachidonic acid and is the only non-steroidal compound known for blocking both of these pathways.

The topical compositions of the present invention include water, an organic peroxide, an alpha hydroxy acid, a moisturizer, an isosorbide and a detergent. The amount of water present in the compositions of this invention may be from about 30 weight percent to about 70 weight percent, based upon the weight of the composition.

Preferably, the amount of water present is from about 35 weight percent to about 70 weight percent.

Organic peroxides which may be included in the topical compositions of the present invention include any pharmaceutically acceptable organic peroxide, such as, for example, benzoyl peroxide, lauroyl peroxide, and carbamide peroxide. Preferably, the organic peroxide is benzoyl peroxide. The amount of organic peroxide present in the compositions of the invention may be from about 1 weight percent to about 20 weight percent, based upon the weight of the composition. Preferably, the organic peroxide is present in an amount from about 2.5 weight percent to about 10 weight percent.

Alpha hydroxy acids which may be included in the topical compositions of the present invention include any pharmaceutically acceptable alpha hydroxy acid, such as, for example, glycolic acid, lactic acid, 2-hydroxydecanoic acid, 2-hydroxystearic acid and malic acid. Preferably, the alpha hydroxy acid is one that is commonly used in topical compositions for treating acne, such as glycolic acid or lactic acid. Most preferably, the alpha hydroxy acid is glycolic acid. The amount of alpha hydroxy acid present in the compositions of the invention may be from about 0.1 weight percent to about 15 weight percent, based upon the weight of the composition. Preferably, the alpha hydroxy acid is present in an amount from about 1 weight percent to about 10 weight percent.

Moisturizers which may be included in the topical compositions of the present invention include any pharmaceutically acceptable moisturizer, such as, for example, sodium pyrollidone carboxylate, glycerin, glycolic acid, propylene glycol and sorbitol. Preferably, the moisturizer is sodium pyrollidone carboxylate. The amount of moisturizer present in the compositions of the invention may be from about 0.5 weight percent to about 20 weight percent, based upon the weight of the composition. Preferably, the moisturizer is present in an amount from about 1 weight percent to about 10 weight percent.

Isosorbides which may be included in the topical compositions of the present invention include any pharmaceutically acceptable isosorbide. Such isosorbides include, for example, dimethyl isosorbide, diethyl isosorbide, and ethylmethyl isosorbide. Preferably, the isosorbide is an alkyl ester of isosorbide, such as dimethyl isosorbide. The amount of isosorbide present in the compositions of the invention may be from about 0.05 weight percent to about 20 weight percent, based upon the weight of the composition. Preferably, the isosorbide is present in an amount from about 0.05 weight percent to about 10 weight percent.

Detergents which may be included in the topical compositions of the present invention include any pharmaceutically acceptable detergent. Such detergents include, for example, sodium potassium lauryl sulfate, cocamidopropyl betaine, sodium cocoylisethionate, and disodium cocoamphopropionate. Preferably, the detergent is sodium potassium lauryl sulfate or cocamidopropyl betaine. The amount of detergent present in the compositions of the invention may be from about 15 weight percent to about 60 weight percent, based upon the weight of the composition. Preferably, the detergent is present in an amount from about 25 weight percent to about 40 weight percent.

The compositions of the present invention also may contain various other ingredients that are commonly included in topical pharmaceutical compositions. Such ingredients include, for example, thickeners, preservatives, binders, wetting agents, and bases.

Thickeners which may be included in the topical compositions of the present invention include any pharmaceutically acceptable thickener. Such thickeners include, for example, cetostearyl alcohol, corn starch, polyethylene glycol, PEG-14M (PEG-14M is available from Amerchol Corp., Edison, N.J.), xanthan gum, cetyl alcohol, bentonite, carbomer, PEG 12, and magnesium aluminum silicate. The thickeners may be present in the compositions of the invention in an amount from about 1 weight percent to about 30 weight percent, based upon the weight of the composition. Preferably, the thickener is present in an amount from about 2 weight percent to about 25 weight percent.

Preservatives which may be included in the topical compositions of the present invention include any pharmaceutically acceptable preservative. Such preservatives include, for example, methylparaben, propylparaben, imidurea, Potassium Sorbate, Phenacetin, and quatemium-15. Preferably, the preservative is methylparaben or imidurea. The amount of preservatives present in the compositions of the invention may be from about 0.05 weight percent to about 1 weight percent, based upon the weight of the composition. Preferably, the preservatives are present in an amount from about 0.1 weight percent to about 0.7 weight percent.

Bases which may be included in the topical compositions of the present invention include any pharmaceutically acceptable base. Such bases include, for example, sodium hydroxide, sodium citrate, sodium acetate, sodium phosphate, and sodium lactate.

Emollients which may be included in the topical compositions of the present invention include any pharmaceutically acceptable emollient. Such emollients include Glyceryl Stearate SE, Glyceryl Stearate, Isopropyl Myrisate, and Aluminium Hydroxide. Preferably the emollients are present in and amount from about 0.1 weight percent to 0.7 weight percent.

The general therapeutic regimen or strategy using the combination of the present invention usually involves once to twice daily applications, preferably twice daily, of the combination to bring the acne under control. Thereafter, depending on the characteristics of the condition, it is possible to maintain clearance by judicious application of the composition less frequently or in lower concentrations.

Controlled studies on a significant number of patients have shown the combined therapy according to the present invention is not only additive, but may truly be synergistic. That is, the combination is more effective than and produces responses not obtained by the usual treatment regimens of benzoyl peroxides, salicylic acids, glycolic acids, and natural anti-oxidant therapies alone. Thus, acne and inflammatory acne clear more rapidly and there is a rapid resolution of scaling, induration, and edema with the combination. Moreover, relapses are delayed and less severe. Significantly, improvement has been demonstrated in conditions which have become refractory to standard of conventional treatments.

It is important to note that Hepes is a compound that has a taurine molecule as one of the major components. Taurine is an important amino acid in the regulation of neurotransmitters, an immune function and in the control of inflammation. Its physiological mechanism is quite complex, but is known to inhibit the lipoxygenase pathway in the arachidonic acid cascade. The target leukotriene has been reported to be LTB4. Another reaction is the regulation of intracellular calcium and the inhibition of protein kinase C. These actions suggest that hepes is a significant agent to reduce cellular inflammation and cellular proliferation.

The key to the present invention is the use of a new molecule, a hepes ester, which makes a whole new entity. All other applications in the literature that describe hepes as an anti-inflammatory agent use a delivery system that is by injection or venous infusion. As explained above in the BACKGROUND OF THE INVENTION, Hepes, Ethane Sulphonic Acid, (not Hepes Oleate) has been used topically for arthritis and rheumatis, as well as some applications for psoriasis, as disclosed by U.S. Pat. Nos. 4,544,656 and 4,753,942 to O'Sullivan. These patents did not disclose the hepes ester, merely hepes without the ester.

The present invention will now be described in more detail with references to the following specific, non-limiting examples. In these cases the composition was extensively evaluated and unless otherwise indicated the combination brought about rapid resolution (control and clearing) within two-three days of twice daily applications.

The following examples were evaluated with the following formula concentrations, with the percentages shown being by weight in the total composition:

### EXAMPLE 1

Using the listed concentrations of the present invention:

| | |
|---|---|
| Benzoyl Peroxide 75% USP | 4.47% |
| Hepes Oleate | 1.0% |
| Deionized Water | 70.56% |
| Glyceryl Stearate SE | 13.0% |
| Propylene Glycol | 4.56% |
| PEG-12 | 2.00% |
| Bentonite | 1.50% |
| Isopropyl Myristate | 0.40% |
| Diazolidinyl urea | 0.30% |
| Hydrocortisone USP | 0.18% |
| Aluminium Hydroxide | 0.20% |
| Carbomer | 0.15% |
| Methyl Paraben | 0.110% |
| Glyceryl Stearate | 0.10% |
| Cetyl Alcohol | 0.10% |
| Sodium Benzoate | 0.10% |
| Phenacetin | 0.10% |
| Propylparaben | 0.03% |

Approximately 10 cases of highly inflammatory acne vulgaris were treated with the composition. Approximately 85% of the group with this condition was brought under control within two to three weeks of twice daily applications.

### EXAMPLE 2

Using the listed concentrations of the present invention:

| | |
|---|---|
| Benzoyl Peroxide 75% USP | 13.40% |
| Hepes Oleate | 2.0% |
| Deionized Water | 61.63% |
| Glyceryl Stearate SE | 13.0% |
| Propylene Glycol | 4.56% |
| PEG-12 | 2.00% |
| Bentonite | 1.50% |
| Isopropyl Myristate | 0.40% |
| Diazolidinyl urea | 0.30% |
| Hydrocortisone USP | 0.18% |
| Aluminium Hydroxide | 0.20% |
| Carbomer | 0.15% |
| Methyl Paraben | 0.110% |
| Glyceryl Stearate | 0.10% |
| Cetyl Alcohol | 0.10% |
| Sodium Benzoate | 0.10% |
| Phenacetin | 0.10% |
| Propylparaben | 0.03% |

Approximately 20 cases of acne vulgaris were treated with the composition. Approximately 90% of the group with this condition was brought under control within a few days to 2 weeks of twice daily applications.

### EXAMPLE 3

Using the listed concentrations of the present invention:

| | |
|---|---|
| Benzoyl Peroxide 75% USP | 17.87% |
| Hepes Oleate | 5.0% |
| Deionized Water | 54.16% |
| Glyceryl Stearate SE | 13.0% |
| Propylene Glycol | 4.56% |
| PEG-12 | 2.00% |
| Bentonite | 1.50% |
| Isopropyl Myristate | 0.40% |
| Diazolidinyl urea | 0.30% |
| Hydrocortisone USP | 0.18% |
| Aluminium Hydroxide | 0.20% |
| Carbomer | 0.15% |
| Methyl Paraben | 0.110% |
| Glyceryl Stearate | 0.10% |
| Cetyl Alcohol | 0.10% |
| Sodium Benzoate | 0.10% |
| Phenacetin | 0.10% |
| Propylparaben | 0.03% |

Approximately 5 cases of severe pastulo-pustular inflammatory acne were treated with the composition. Approximately 75% of the group with this condition was brought under control within three to six weeks with two to three applications daily.

In these examples, the patients chosen ranged in ages from 10 to 55 years old and ranged in skin tones form very light to very dark complexions.

As such, an invention has been disclosed in terms of preferred embodiments thereof which fulfill each and every one of the objects of the present invention as set forth hereinabove, and provide a new and useful composition and method for treating acne including anti-inflammatory Hepes Oleate of great novelty and utility.

Of course, various changes, modifications and alterations in the teachings of the present invention may be contemplated by those skilled in the art without departing from the intended spirit and scope thereof.

As such, it is intended that the present invention only be limited by the terms of the appended claims.

1. A topical composition for the treatment of acne consisting essentially of:
a) deionized water present in the amount from about 25% to 70%, by weight, of the composition;
b) an anti-acne agent present in the amount from about 1%, by weight, to about 20.0%, by weight, of the composition; and
c) anti-inflammatory Hepes Oleate present in the amount from about 0.5% to 20.0%, by weight, of the composition.

2. The composition of claim 1, further including a reducing agent in the amount of from about 0.05% to 0.25%, by weight, of the composition.

3. The composition of claim 3, wherein said reducing agent comprises Hydrocortisone USP.

4. The composition of claim 1, further including a moisturizer selected from the group consisting of sodium pyrollidone carboxylate, glycerin, propylene glycol, sorbitol and mixtures thereof and present in an amount from about 0.1% to 10.0%, by weight, of the composition.

5. The composition of claim 4, further including an emulsifier selected from the group consisting of PEG-12, Glyceryl Stearate SE, Glyceryl Stearate, Isopropyl Myristate, Aluminum Hydroxide and mixtures thereof and present in the amount from about 0.1% to 20.0%, by weight, of the composition.

6. The composition of claim 5, further including thickeners selected from the group consisting of Bentonite, Cetyl Alcohol, Carbomer and mixtures thereof and present in an amount from about 0.1% to 10.0%, by weight, of the composition.

7. The composition of claim 6, further including a pH balancer selected from the group consisting of Potassium Hydroxide or Sodium Hydroxide present and in the amount from about 0.01% to 0.15%, by weight, of the composition.

8. The composition of claim 7, further including anti-microbials and preservatives selected from the group consisting of Sodium Benzoate, Potassium Sorbate, Phenacetin, Methyl Paraben, Propylparaben, Imidazolidinyl Urea and mixtures thereof and present in the amount from about 0.05% to 10.0%, by weight, of the composition.

9. The composition of claim 3, wherein said benzoyl peroxide is present in an amount from about 2.5% to 10.0%, by weight, of the composition.

10. The composition of claim 1, wherein said deionized water is present in an amount from about 30% to 70.0%, by weight, of the composition.

11. The composition of claim 3, wherein said Hydrocortisone USP is present in an amount from about 0.05% to 0.25%, by weight, of the composition.

12. A method of suppressing inflammation in papulo-pustular inflammatory acne using the composition of claim 3, wherein said Hepes Oleate and benzoyl peroxide being present in synergistic effective amounts which are effective to suppress said inflammation and control and clear said acne.

13. The method of claim 12, wherein said Hepes Oleate is present in the amount from about 1% to 5%, by weight, of the composition.

14. The composition of claim 4, wherein said moisturizer is present as propylene glycol.

15. The composition of claim 1, further including an emulsifier comprising a mixture of PEG-12, Glyceryl Stearate SE, Glyceryl Stearate, Isopropyl Myristate, Aluminum Hydroxide.

16. The composition of claim 6, wherein said thickener is a mixture of Bentonite, Cetyl Alcohol, Carbomer.

17. The composition of claim 7, wherein said pH balancer is Potassium Hydroxide.

18. The composition of claim 8, wherein said anti-microbials and preservatives comprise a mixture of Sodium Benzoate, Potassium Sorbate, Phenacetin, Methyl Paraben, Propylparaben and Imidazolidinyl Urea.

19. The composition of claim 1, further comprising a stabilizer.

20. A method for treating a person afflicted with acne which comprises applying to an affected area of the person's skin, in a therapeutically effective amount, a composition consisting essentially of:
a) water;
b) benzoyl peroxide in an amount from about 2.5% to 10.0%, by weight, of the composition;
c) Hydrocortisone USP in an amount from about 0.05% to 0.25%, by weight, of the composition;
d) Hepes Oleate in an amount from about 0.5% to 20.0%, by weight, of the composition;
e) a moisturizer selected from the group consisting of sodium pyrollidone carboxylate, glycerin, propylene glycol, sorbitol and mixtures thereof and present in an amount from about 0.1% to 10.0%, by weight, of the composition;
f) an emulsifier selected from the group consisting of PEG-12, Glyceryl Stearate SE, Glyceryl Stearate, Isopropyl Myristate, Aluminum Hydroxide and mixtures thereof and present in an amount from about 0.1% to 20.0%, by weight, of the composition;
g) thickeners selected from the group consisting of Bentonite, Cetyl Alcohol, Carbomer and mixtures thereof and present in an amount from about 0.1% to 10.0%, by weight, of the composition;
h) a pH balancer comprising Potassium Hydroxide; and
i) anti-microbials and preservatives selected from the group consisting of Sodium Benzoate, Potassium Sorbate, Phenacetin, Methyl Paraben, Propylparaben, Imidazolidinyl Urea and mixtures thereof and present in an amount from about 0.05% to 10.0%, by weight, of the composition.
